(19) United States
(12) Patent Application Publication
(10) Pub. No.: US 2006/0020290 A1
(43) Pub. Date: Jan. 26, 2006

(54) Medical device with memory upload trigger

(75) Inventor: Paul J. Degroot, Brooklyn Park, MN (US)

Correspondence Address: MEDTRONIC, INC., 710 MEDTRONIC PARK, MINNEAPOLIS, MN, 55432-9924, US

(73) Assignee: Medtronic, Inc.

(21) Appl. No.: 10/945,575

(22) Filed: Sep. 21, 2004

Related U.S. Application Data
(60) Provisional application No. 60/589,249, filed on Jul. 20, 2004.

(51) Int. Cl. A61N 1/00; A61K 9/22

(52) U.S. Cl. 607/2; 128/903; 604/890.1

(57) ABSTRACT

A medical device includes an alert to signal that the amount of stored data is approaching the capacity of the memory in the medical device, and the data must be uploaded to avoid losing the data. A patient perceptible alert may trigger the patient to influence immediate/timely action for data transmission. A silent alert may trigger automatic data transmission, or trigger a medical support network to autonomously conduct transmission of the data.

### CROSS-REFERENCE TO RELATED APPLICATION(S)

This application claims the benefit of U.S. Provisional Application No. 60/589,249 filed on Jul. 20, 2004, for “Device Memory Upload Trigger” by P. DeGroot.

### INCORPORATION BY REFERENCE

The aforementioned U.S. Provisional Application No. 60/589,249 is hereby incorporated by reference in its entirety.

### BACKGROUND OF THE INVENTION

The present invention relates to an improved medical device that minimizes loss of data stored in the memory of the medical device.

Various medical devices gather and store data in a memory located within the device. The data that is gathered is useful for proper patient management, or it may be important for clinical investigations.

Implantable medical devices (IMDs), such as pacemakers, cardioverter/defibrillators, drug delivery devices, and nerve stimulators are designed to be as small as possible while encasing all necessary components. This necessitates the use of low power components, so that the IMDs can be operated for extended periods of time without battery replacement. Current IMDs, therefore, make use of low voltage, low current memory components that have limited storage capacity.

A problem experienced by those that manage patients or perform clinical investigations on patients with IMDs is that data gathered by the IMDs is routinely lost. Patients are regularly scheduled to upload the data stored in their IMDs. However, in many instances, the amount of data gathered exceeds the limited storage capacity of the IMD's memory. As the memory overfills, it is typically configured to write over the earliest data stored in the device memory. This continues until the data is uploaded, at which time the memory may be cleared.

In many cases patients are not aware of relevant clinical events as they occur. Data related to these events are continuously gathered and stored by the IMD, but the patient is not aware that the amount of stored data is approaching or has reached the capacity of the memory. As a result, data may be lost, with neither the patient nor a caregiver being aware of the quantity or content of the data.

### BRIEF SUMMARY OF THE INVENTION

The present invention is a system that reduces the loss of data stored in a medical device. A processor within the medical device initiates an alert when the amount of stored data approaches the capacity of the memory of the device. The alert signals the need to transmit the data stored in the memory of the medical device to a medical support network thus enabling a safety alert to preserve data.

### BRIEF DESCRIPTION OF THE DRAWINGS

FIG. 1 is a schematic diagram of a representative system utilizing the present invention.

FIG. 2 is a schematic diagram of a representative system utilizing the present invention.

FIG. 3 is a block diagram of an implantable medical device.

FIG. 4 is a block diagram of a monitor.

FIG. 5 is a block diagram of a patient management network.

FIG. 6 is a flowchart illustrating the present invention.

### DESCRIPTION

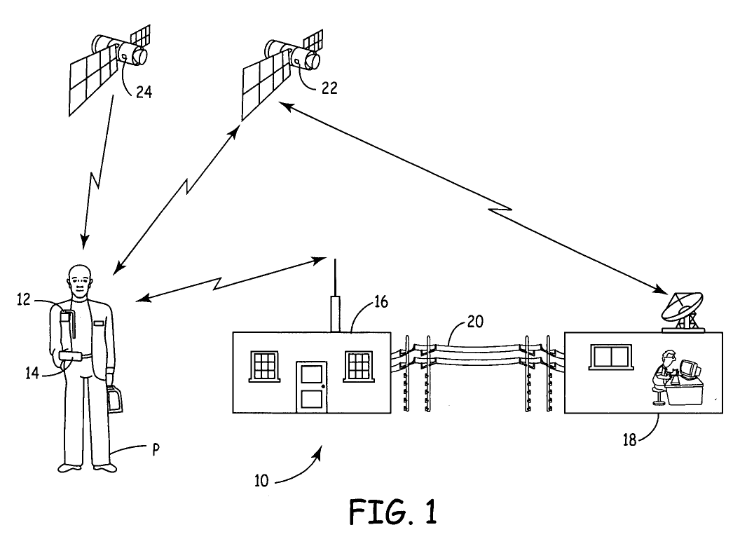

FIG. 1 illustrates a representative system by which data from a medical device is gathered by a medical support network. System 10 includes patient P with implantable medical device (IMD) 12, portable external device 14, communications link transceiver 16, medical support network 18, phone lines 20, communications link satellite 22, and global positioning system (GPS) satellite 24.

IMD 12 is shown here as an implantable medical device, however, the medical device may also be external rather than implantable. External device 14 is portable and carried with patient P and communicates with IMD 12 by wireless signals. External device 14 communicates with communications link transceiver 16, again, by wireless signals. Transceiver 16, in turn, communicates with medical support network 18 via phone lines 20. However, any of a number of forms of communication of data may be used. External device 14 may communicate with medical support network 18 via communications link satellite 22. Lastly, external device 14 may also communicate with GPS satellite 24.

In operation, physiological data of patient P and operational data of IMD 12 are gathered and stored in the memory of IMD 12. Typically, patient P is scheduled to transmit the data stored in the memory of IMD 12 at regular intervals. The transmission of data occurs remotely through this system such that patient P is not required to make an in-office visit to a caregiver.

Triggering data upload from IMD 12 may occur by any of a number of different ways. Patient P may initiate uploading of data via external device 14. Communication between medical device 12 and external device 14 is through wireless signals such as radio frequency (RF) signals. External device 14 subsequently transmits the data to medical support network 18 via alternative pathways. In a first pathway, the data is transmitted to communications link transceiver 16 by wireless communication. The data is then sent to medical support network 18 over phone lines 20. In a second pathway, the data is transmitted by wireless communication to medical support network 18 via communications link satellite 22.

Alternatively, uploading of data may be initiated via a signal generated from medical support network 18. The signal can be sent to external device 14 through either of the pathways described above.

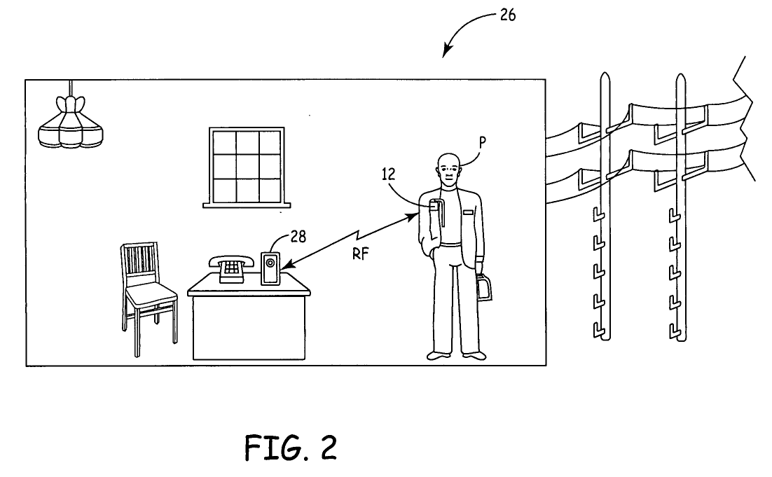

As shown in FIG. 1, portable external device 14 carried or worn by patient P. However, as shown in system 26 in FIG. 2 monitor 28 may also function as an external device in place of or in addition to portable external device 14. Only part of system 26 is shown and described. Here, monitor 28 is utilized instead of external device 14, but device 14 and monitor 28 provide the same functions. Monitor 28 utilizes telemetry with range suitable for communicating over appropriate distances, and is kept in any area where patient P will regularly come into range of monitor 28. The telemetry protocol defines the distance and may require close proximity to a programming head to facilitate indicative communications to longer distances of a few feet to e.g., 30 feet of course, when properly supported telemetry would be facilitated over even great distances and is not meant as a limiting factor. In this embodiment, communication between stationary device 28 and medical support network 18 is, for example, via a hard-wired phone line. Uploading of data from medical device 12 is initiated as described above for external device 14. However, transmission of data from IMD 12 using system 26 only occurs while patient P is within range of monitor 28.

The memory of IMD 12 has a limited capacity for storing data. The amount of stored data may surpass the memory's capacity before patient P is scheduled for uploading data from IMD 12. As the data overfills the memory, new data typically writes over the initial data, and the initial data is lost.

IMD 12 is a device such as a pacemaker or defibrillator that is implanted in patient P and is capable of providing life-saving and life-enhancing cardiac therapies. These therapies may include providing pacing pulses or defibrillation shocks to the heart of patient P or providing drug therapy. IMD 12 also records useful data related to the condition of patient P and IMD 12 and periodically provides that data to a caregiver. In addition, IMD 12 is capable of detecting the occurrence of an event that satisfies predefined alert criteria. The alert criteria pertain to either a clinically-relevant event, such as an arrhythmia, or to the functioning of IMD 12 and are guidelines determining when to attract the patient's and/or caregiver's attention. Once an event is detected that satisfies an alert criterion, IMD 12 is capable of providing a patient perceptible alert and/or a silent alert. A patient perceptible alert notifies patient P of a triggered alert criterion via, for example, an audible tone (or mechanical vibration, electric shock, olfactory signal, etc.) from IMD 12, device 14, or monitor 28. A silent alert is a caregiver or network 18 notification of a triggered alert criterion via system 10 or 26.

Monitor 28 is a device, such as the Medtronic CareLink monitor, intended for use in a patient's home that is capable of receiving data from the patient's implanted device via telemetry and transmitting this information via phone lines or other communication link to a private network, which transfers the data to network 18. In one embodiment, the private network is the IP Link service from MCI, which provides a private, secure, and reliable connection.

Network 18 utilizes secure computer servers that collect, process, and store data sent from monitor 28 or device 14. This information is available to patient P and a caregiver through patient management web clients. Patient management web clients are computer systems with a browser capable of viewing web pages on the World Wide Web.

There are at least three follow-up scenarios in which a caregiver can interact with IMD 12 to monitor the condition of patient P and IMD 12: standard follow-up, remote follow-up, and ambulatory follow-up. Standard follow-up is a scheduled face-to-face interaction between patient P and a caregiver in order to check the patient's health/status and the functioning of IMD 12. Typically, the standard follow-up occurs every three to six months. System 10 or 26 of the present invention reduces the number of standard follow-ups that need to take place. The remote follow-up is a scheduled electronic transmission of the data stored in IMD 12 to a caregiver in order to check the health of patient P and the functioning of IMD 12. Similar to the standard follow-up, the remote follow-up typically occurs every three to six months. The remote follow-up is enabled by use of monitor 28 or device 14 and network 18. The ambulatory follow-up is an unscheduled and IMD-initiated electronic transmission of the data stored in IMD 12 to a caregiver in order to alert the caregiver to the occurrence of an event that satisfies the alert criteria to allow a caregiver to check the status of patient P and the functioning of IMD 12. Standard follow-ups may be time-consuming, inconvenient, and often unnecessary for both patient P and the caregiver. Ambulatory follow-ups, however, can be provided by system 10 or 26 of the present invention to provide many benefits.

System 10 or 26 of the present invention is capable of providing remote follow-ups as well as silent alerts for ambulatory follow-ups. Communication between the various components of system 10 or 26 will now be described. Either upon the detection of the amount of stored data approaching the capacity of the memory, or at a scheduled time, IMD 12 is interrogated by monitor 28 or device 14 over a wireless telemetry system utilizing RF signals. Monitor 28 or device 14 then communicates the data to network 18 from the home of patient P. Data is then displayed to the caregiver or patient P using patient management web clients utilizing the standard World Wide Web (“WWW”) secured communication protocol (i.e. SSL).

System 10 or 26 increases the caregiver's efficiency in managing, from his or her office, patient P from the patient's home; while also increasing the caregiver's ability to identify and react to clinical conditions and disease management issues. System 10 or 26 reduces reliance on the memory of patient P to upload data before it is lost and on the awareness of patient P that clinical events are occurring.

The present invention can also be utilized in clinical trial studies. It is useful to have as much data as possible retained during clinical trials to ensure that important data is not lost. An alert indicating that the amount of stored data is approaching the capacity of the memory reduces the loss of data.

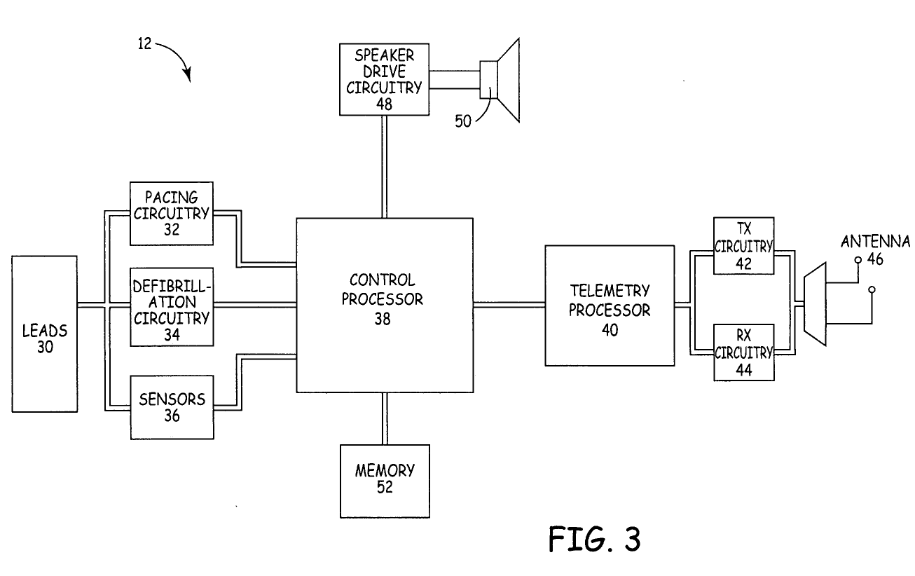

FIG. 3 is a block diagram of IMD 12 of system 10 or 26 of the present invention. Although it is recognized that system 10 or 26 can be used with any type of implantable medical device, a specific example will now be provided in which IMD 12 is an implantable cardioverter/defibrillator (ICD). IMD 12 includes leads 30, pacing circuitry 32, defibrillation circuitry 34, sensors 36, control processor 38, telemetry processor 40, transmitter circuitry 42, receiver circuitry 44, antenna 46, speaker drive circuitry 48, speaker 50, and memory 52.

Control processor 38′ is the primary controller for the overall operation of IMD 12. Specifically, control processor 38 controls pacing circuitry 32 and defibrillation circuitry 34 to provide therapeutic electrical pulses to leads 30. Leads 30 are preferably implanted within the heart of patient P and provide an electrically conductive path for the pulses to selected locations within the heart. In addition, leads 30 can be used by sensors 36 to detect cardiac signals in the heart. Together, leads 30 and sensors 36 are a means for sensing physiological parameters. These cardiac signals are conducted through leads 30, detected by sensors 36, and then provided to control processor 38. Control processor 38 saves the signals in memory 52, which is, for example, random access memory (RAM).

Control processor 38 is capable of analyzing the cardiac signals received from sensors 36 and monitoring the condition of IMD 12 to determine, among other things, whether the amount of data stored in memory 52 is approaching the capacity of memory 52. Control processor 38 is a means for activating an alert. If control processor 38 determines that the amount of data stored in memory 52 is approaching the capacity of memory 52, it then decides, based upon caregiver selectable alert settings, what type of an alert should be provided. If the caregiver selectable alert settings instruct control processor 38 to provide a patient alert, an alert signal is generated and sent to speaker drive circuitry 48. Speaker drive circuitry 48 provides the necessary electrical signal to speaker 50 to generate an audible sound that alerts patient P that the amount of stored data is approaching the capacity of memory 52, and data from IMD 12 should be uploaded.

On the other hand, if caregiver selectable alert settings instruct control processor 38 to provide a silent alert, then an alert signal is generated and sent to telemetry processor 40. The circuitry required to generate, initiate, or activate an alert may be referred to as alert circuitry. Telemetry processor 40 then controls transmitter circuitry 42 to emit an RF alert signal that is transmitted wirelessly over antenna 46. The alert signal alerts external device 14 (FIG. 1) or monitor 28 (FIG. 2) that IMD 12 is set to communicate with it, provided that external device 14 or monitor 28 is within the telemetry range of IMD 12. Transmitter circuitry 42 is also a means for transmitting data stored in the memory. In this way, IMD 12 is capable of initiating communication with external device 14 or monitor 28 to inform device 14 or monitor 28 that the amount of stored data is approaching the capacity of memory 52.

Control processor 38 may detect that the amount of stored data is approaching the capacity of memory 52 by any of a number of methods. In a representative embodiment, control processor 38 is instructed to alert when data is saved at a specific address in memory 52. The specific address correlates to the amount of stored data that approaches the capacity of memory 52.

It is recognized that IMD 12 could be utilized to provide an alert signal in response to any detectable event to avoid loss of recorded data. Other events may include therapy delivery, arrhythmias, heart failure, system integrity, ischemia, or a neurologic or neurocardiogenic event such as an epileptic seizure.

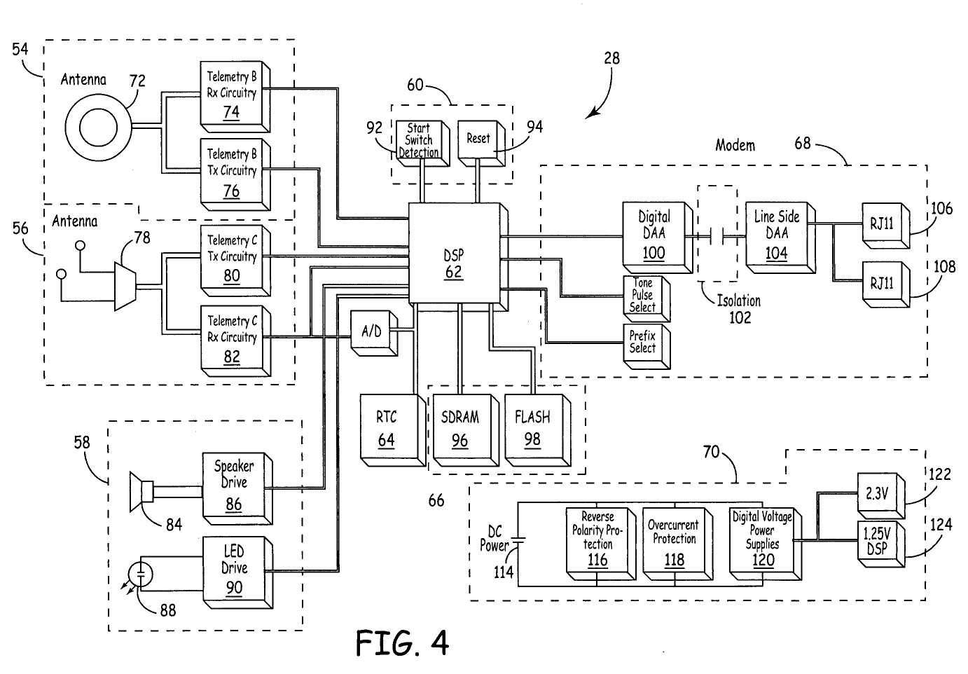

FIG. 4 is a block diagram of monitor 28 of system 26 of the present invention. All or some of the same components are also used by external device 14 of system 10. Monitor 28 includes communication system 54, wireless communication system 56, patient alert module 58, control switches 60, digital signal processor (“DSP”) 62, real-time clock (“RTC”) 64, memory 66, modem 68, and power supply 70. Short-distance communication system 54 includes antenna 72, receiver circuitry 74, and transmitter circuitry 76. Wireless communication system 56 includes antenna 78, transmitter circuitry 80, and receiver circuitry 82. Patient alerts include speaker 84, speaker drive 86, light-emitting diodes (LEDs) 88, and LED drive 90. Control switches 60 include start switch detection 92, and reset 94. Memory 66 includes SDRAM 96 and flash memory 98. Modem 68 includes digital data access arrangement (“digital DAA”) 100, isolation 102, line side DAA 104, RJ11 ports 106 and 108, tone/pulse select 110, and prefix select 112. Power supply 70 includes DC power 114, reverse polarity protection 116, overcurrent protection 118, digital voltage power supplies 120, and DC outputs 122 and 124.

Monitor 28 is preferably located within the home of patient P or otherwise mentioned within proximity to the patient P. Monitor 28 is capable of wireless communication with IMD 12 via wireless communication system 56 over distances facilitated by the selected telemetry protocol. Communication system 54 uses an intermediary device, such as a programming head, placed proximate the IMD to communicate (e.g., via inductive coupling). Wireless communication system 56 permits communication over longer ranges, e.g., from a few feet to 30 feet, or even significantly greater. Short-distance communication system 54, which communicates over a distance of up to about 25 feet, is also provided to enable communication with implantable medical devices that utilize short-distance head-based communication.

Patient alert module 58 includes speaker 84 coupled to speaker drive 86 and LEDs 88 coupled to LED drive 90. Speaker drive 86 and LED drive 90 are both controlled by DSP 62. Speaker drive 86 and speaker 84 serve two functions: to generate tones to indicate an alert condition, and to make modem 68 audible. LEDs 88 are used as visual indicators to give status indications to patient P or a caregiver during an interrogation and modem connection. LEDs 88 also alert patient P to power status and completion of uploaded data to the server. Switches 60 provide buttons that allow patient P to interact with monitor 28. Switches 60 include start switch detection 92 and reset 94. Start switch detection 92 allows patient P to instruct monitor 28 to begin an interrogation of IMD 12. Reset 94 allows patient P to reset monitor 28 to factory defined settings.

Real-time clock 64 is provided in monitor 28 to keep track of the time. Both IMD 12 and monitor 28 keep track of the time so that communication can take place at predetermined times. In order to save battery power in IMD 12, the telemetry system of IMD 12 does not remain active at all times. Instead, IMD 12 and monitor 28 have predefined communication times during which routine communication can take place. However, as described above, system 10 and 26 also include the capability of IMD 12 initiated communication at any time when the amount of stored data approaches the capacity of the memory.

SDRAM 96 of memory 66 is used to store interrogation data from IMD 12 as well as program code and other program-related data. Flash memory 98 is used to store program data and any parameters that need to be stored in non-volatile memory (e.g. phone numbers). DSP 62 boots from flash 98.

Power supply 70 provides DC power to monitor 28. The function of power supply 70 should be easily understood by one skilled in the art and therefore will not be described in further detail.

Monitor 28 is an interrogation and data transfer tool used with IMD 12. Monitor 28 offers the capabilities to patient P, a caregiver, and service personnel of remote interrogations, data processing, reporting and follow-up to be performed when the patient is at home and the caregiver is in the clinic or a location that has web-enabled capability. This remote feature allows for reduced travel and waiting time, providing prompt care to patients and better efficiencies to caregivers. It also enables caregivers to better manage patients and still maintain the quality of care that is warranted in the marketplace. Furthermore, monitor 28 allows field representatives to increase their productivity, provide equal or better service to existing and new customers worldwide, and control costs for providing the services. The increased productivity is obtained by reducing the time required for manufacturer-assisted follow-up. Monitor 28 interrogates IMD 12 and stores the data. Further, monitor 28 collaborates with network 18 to confirm the establishment of a connection with network 18, performs any required file translation functions necessary for data transfer, and executes the data file transfer and then collaborates with network 18 to confirm that the data file transfer was successful. If external device 14 is utilized, communication between network 18 and device 14 may be via satellite link 22 such as described in U.S. Pat. No. 6,292,698, assigned to Medtronic, Inc.

Now that the structures of IMD 12 and monitor 28 have been described, the communications between IMD 12 and monitor 28 will be described. As explained above, there are at least three primary follow-up scenarios in which a caregiver and IMD 12 interact: standard follow-up, remote follow-up, and ambulatory follow-up. Of these, a remote follow-up and an ambulatory follow-up utilize monitor 28 as one of the communication links between IMD 12 and the caregiver. A remote follow-up occurs every three to six months at a scheduled time. An ambulatory follow-up, on the other hand, may occur when IMD 12 detects that the amount of stored data is approaching the capacity of memory 52. Thus, the primary difference between the two follow-up procedures is that in an ambulatory follow-up procedure IMD 12 must initiate communication with monitor 28, since the communication is not scheduled. A remote follow-up, on the other hand, is scheduled and expected by IMD 12, and therefore is initiated by monitor 28. This procedure also satisfies current FCC regulations, which indicate that an implantable medical device operating in the MICS band may not initiate communications unless a “medical implant event” occurs. (Title 47 of the Code of Federal Regulations, Part 95.628.) The FCC has further defined the event as an occurrence that necessitates data exchange in order to maintain patient safety.

Once communication has been established, monitor 28 typically performs a full interrogation of IMD 12. Control processor 38 of IMD 12 reads the desired data from memory 52 and then provides it to telemetry processor 40. Telemetry processor 40 and transmitter circuitry 42 transform the data to an RF signal that is wirelessly transmitted by antenna 46 to monitor 28. Monitor 28 receives the wireless transmission of data through antenna 78 and wireless receiver circuitry 82. Receiver circuitry 82 then provides the data to DSP 62, which stores the data in SDRAM 96. After all desired data has been received; the communication between monitor 28 and IMD 12 is closed.

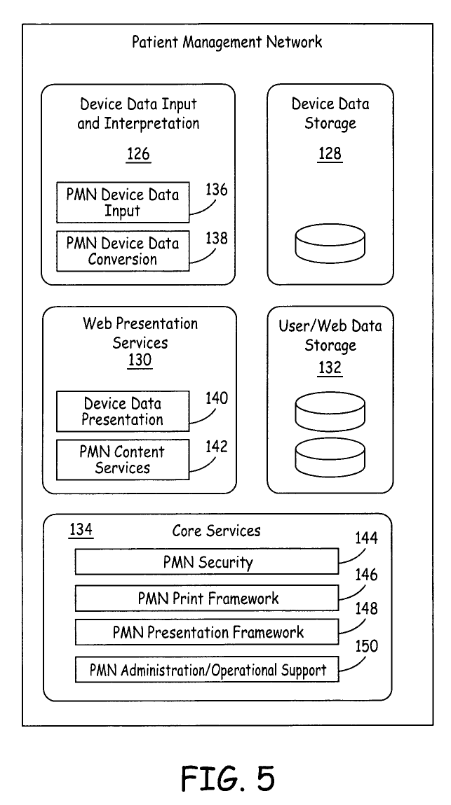

FIG. 5 is a block diagram of network 18 of system 10 or 26 of the present invention. Network 18 includes device data input and interpretation 126, device data storage 128, web presentation services 130, user/web data storage 132, and core services 134. Device data input and interpretation 126 includes device data input 136 and device data conversion 138. Web presentation services 130 include device data presentation 140 and patient management network (“PMN”) content services 142. Core services 134 include PMN security 144, PMN print framework 146, PMN presentation framework 148, and PMN administration/operational support 150.

Network 18 utilizes secure computer servers that collect, process and store data sent from monitor 28. This data is then made available to patient P and a caregiver through Internet accessible web sites that are personalized for their particular needs.

After monitor 28 has completed a full interrogation of IMD 12, it then transfers the data over a telephone line to link 16. Link 16 is, for example, MCI's IP Link private network. Link 16 allows monitor 28 to remotely access network 18 over a private, secure, and reliable connection. Network 18, which consists of secure computer servers, receives the data from monitor 28 (over link 16) and into device data input and interpretation 126 and more specifically through PMN device data input 136, which, in one embodiment, includes a dedicated router. The data is then processed by PMN device data conversion 138 and stored in device data storage 128. Further processing is performed by web presentation services 130 to turn the raw device data into viewable portable document format (“PDF”) documents, graphs, tables, etc. and also to create client and patient personalized web sites which are accessed by a patient browser and caregiver browser. This data is then stored in user/web data storage 132. Additionally, core services 134 are performed by network 18 to provide PMN security 144, PMN print framework 146, PMN presentation framework 148, and PMN administration/operational support 150.

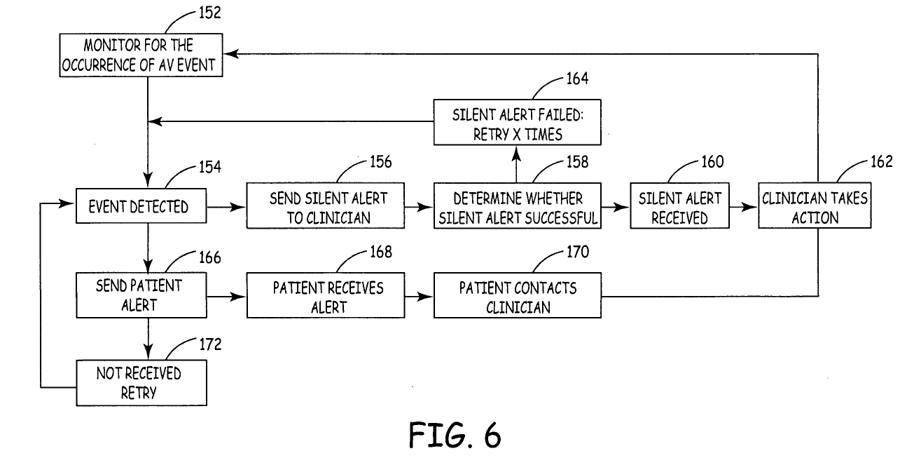

FIG. 6 is a flow diagram illustrating one embodiment of a method of sending an alert signal from IMD 12 upon the amount of stored data approaching the capacity of memory 52. IMD 12 begins by monitoring for the occurrence of the amount of stored data approaching the capacity of memory 52 (step 152). When this occurrence is detected (step 154), the system selects who should first be notified based upon predefined alert criteria. This step is preferably performed by IMD 12 but may also be performed by device 14, monitor 28, or network 18. If IMD 12 decides to attempt a silent alert to a caregiver (step 156), IMD 12 wirelessly transmits an alert signal to monitor 28 or external device 14. If monitor 28 or device 14 receives the alert signal, a full interrogation of IMD 12 is performed as defined above and the session is closed. Monitor 28 or device 14 then transfers the data to network 18, which informs the caregiver. The system then determines whether the silent alert was successfully communicated to the caregiver (step 158).

Various methods of determining the success of the silent alert may be used. For example, monitor 28 or device 14 can provide a verification signal to IMD 12 after the data is successfully transferred to network 18, or the caregiver can provide a verification signal to network 18 that is sent through system 10 or 26 to IMD 12, etc. If system 10 or 26 determines that the silent alert has been received (step 160), it knows that the caregiver will take the necessary action (step 162). If system 10 or 26 determines that the silent alert failed (step 164) (for example, if no verification signal is received within a predetermined amount of time), IMD 12 assumes that the alert was not successfully communicated. As a result, IMD 12 repeats the attempted transmission a predetermined number of times (steps 154, 156, 158, and 164). Because the most frequent cause of a failed transmission is that IMD 12 is not in range of monitor 28, IMD 12 waits for a specified amount of time, such as three hours, before retrying the transmission. So, for example, IMD 12 will continue attempting communication every three hours for up to three days for a total of twenty four times.

If repeated attempts to transmit the alert signal are unsuccessful, IMD 12 will then switch to the backup alarm. The backup alarm may be, for example, patient perceptible signals generated by speaker drive 48 and speaker 50. Thus, after repeated unsuccessful attempts to wirelessly transmit the alert signal (steps 154, 156, 158, and 164), a patient perceptible signal is provided (step 166). If patient P receives the alert signal, IMD 12 can be designed with a user response device, such as a magnetic sensor, a wireless telemetry input device, or other manual input means, which allows patient P to verify that he or she has received the alert. To do so, system 10 or 26 detects when a full interrogation of IMD 12 has taken place, and recognizes at that point that the patient alert has been received.

In alternate embodiments, the alert may also be triggered by situations in which the wireless transmission is considered a failure, such as: when the alert signal is not received by monitor 28 or device 14, when the interrogation of IMD 12 by monitor 28 or device 14 is incomplete, when network 18 does not receive the data, or when the caregiver does not acknowledge the alert after being informed by network 18.

System 10 or 26 of the present invention provides a caregiver selectable user interface in which the caregiver can set the caregiver selectable alert settings of system 10 or 26 to perform as desired. These settings define the alert criteria that are used by IMD 12 to determine whether or not an alert should be sent, and whether a silent alert or a patient alert should be sent. Thus, system 10 or 26 provides the caregiver with a user interface in which he or she can select which events should initiate a silent alert, a patient alert, both alerts, or no alert at all.

System 10 or 26 not only allows the caregiver to enable or disable the alert conditions, but also allows the caregiver to select the response to the condition. If the caregiver selects the alert mode to be “audible,” the alert method is set as a patient alert. If the caregiver selects the alert mode to be “silent,” the alert method will be a silent alert. Finally, if the user selects the alert mode to be “audible+silent,” both methods of notification will be used.

System 10 or 26 of the present invention provides an alert for memory upload that can be provided to various people based upon caregiver selectable alert settings, thereby increasing the safety and quality of life of a patient. It provides a system and method for transferring data automatically without any interaction by the patient. It reduces the number of times that the patient must travel to a clinic for memory upload, thereby reducing the burden on the patient and increasing the efficiency of the caregiver.

The system of the present invention includes a number of patient alert methods including a speaker in IMD 12 and a speaker and LEDs in monitor 28. Other known types of patient alerts may also be used including muscle stimulation, vibration, or olfactory stimulation (in an external device such as monitor 28). Similarly, various means of alerting the caregiver (or any other person) are contemplated. An alert may be provided to a device worn or nearby a caregiver such as a telemetry enabled watch, home PC, public access transponder, WiFi/Bluetooth network, telephone, pager, cell phone, or displayed on a programmer during the next interrogation. Alternatively, the alert could be provided to a call center from monitor 28 or network 18, the call center having an operator who would contact the caregiver. The alerts may include all information from the interrogation of IMD 12, or it may be simply a message informing the caregiver to check the caregiver website. Furthermore, it is recognized that this alert may be provided for any other device in the system or a system in communication with any device in the system.

Although systems 10 and 26 of the present invention have been described with reference to preferred embodiments, workers skilled in the art will recognize that changes may be made in form and detail without departing from the spirit and scope of the invention.

1. A medical device comprising:
a sensor for gathering data;
memory for storing the data;
a processor for detecting an amount of data stored in the memory and triggering an alert when the amount of stored data approaches a capacity of the memory; and
a transceiver for transmitting the data stored in the memory following the alert.

2. The medical device of claim 1 wherein the alert includes patient perceptible signals.

3. The medical device of claim 2 wherein the patient perceptible signals include at least one of: an auditory alert, a vibratory alert, a visual alert, an olfactory alert, an electrical alert, a magnetic alert, and an electromagnetic alert.

4. The medical device of claim 1 wherein the processor triggers an external device to generate patient perceptible signals.

5. The medical device of claim 4 wherein the external device that generates patient perceptible signals includes one of: a pager, an alarm, a cell phone, and a medical device monitor.

6. The medical device of claim 1 wherein the transceiver sends the alert to a network when the amount of stored data approaches the capacity of the memory.

7. The medical device of claim 6 wherein the transceiver transmits the data upon receiving from the network, a signal to initiate transmission of the data.

8. The medical device of claim 6 wherein the processor, in response to a signal from the network, triggers a patient perceptible signal to indicate readiness for data transmission.

9. The medical device of claim 6 wherein the processor causes a patient perceptible signal to be generated if the processor has not received confirmation that the network received the alert.

10. The medical device of claim 1 wherein the processor triggers the alert at regular intervals until the data is transmitted.

11. The medical device of claim 1 wherein the transceiver transmits data to a medical support network.

12. The medical device of claim 1 wherein the transceiver transmits data to an external device.

13. An implantable medical device comprising:
means for delivering therapy;
memory for storing data related to the therapy;
means for activating an alert when an amount of stored data approaches a capacity of the memory; and
means for transmitting the data stored in the memory following the alert.

14. The implantable medical device of claim 14 and further comprising:
means for sensing physiological parameters, which are stored as data in the memory.

15. The implantable medical device of claim 13 wherein the alert is in a patient perceptible form.

16. The implantable medical device of claim 13 wherein the alert is detected by the means for transmitting.

17. The implantable medical device of claim 13 wherein the alert is transmitted to an external device.

18. The implantable medical device of claim 13 wherein the alert is transmitted to a medical support network.

19. The implantable medical device of claim 13 wherein the means for transmitting is a communication system with a range of up to about 30 feet.

20. The implantable medical device of claim 13 wherein the means for activating an alert is a processor.

21. The implantable medical device of claim 13 wherein the therapy includes one of: electric stimulation therapy and drug therapy.

22. A system for acquiring data comprising:
an implantable medical device comprising:
memory for storing the data;
a processor for controlling components of the implantable medical device;
alert circuitry that is activated by the processor when an amount of stored data approaches a capacity of the memory; and
a transceiver for transmitting signals and the data and receiving signals;
an external device in at least intermittent communication with the implantable medical device, the external device receiving the data and signals transmitted by the transceiver; and
a medical support network in communication with the external device.

23. The system of claim 22 and further comprising:
a phone link for communication between the external device and the medical support network.

24. The system of claim 22 and further comprising:
a communications satellite link for communication between the external device and the medical support network.

25. The system of claim 22 wherein the alert circuitry signals the external device to initiate data transmission.

26. The system of claim 22 wherein the alert circuitry signals the transceiver to transmit the data to the external device.

27. The system of claim 22 wherein the alert circuitry applies a patient perceptible signal to alert a patient to initiate data transmission to the external device.

28. The system of claim 22 wherein the alert circuitry signals the medical support network via the external device, and the medical support network initiates data transmission via the external device.

29. The system of claim 22 wherein the transceiver is a telemetry transceiver.

30. The system of claim 22 wherein the external device is portable.

31. The system of claim 22 wherein the external device is a monitor.

32. The system of claim 22 wherein the alert circuitry signals a patient as back-up to initiate data transmission.

33. A method of preserving stored data in a medical device, the method comprising:
storing data related to the implantable medical device and physiological parameters of the patient in a memory of the implantable medical device;
detecting that an amount of stored data approaches a capacity of the memory; and
providing an alert indicating that the amount of stored data approaches the capacity of the memory so that the data is uploaded from the memory before the data is overwritten.

34. The method of claim 33 wherein detecting that the amount of stored data approaches the capacity of the memory further comprises:
detecting when data is saved at a specific address, the specific address correlating to the amount of stored data that approaches the capacity of the memory.

35. The method of claim 33 and further comprising:
repeating the alert if the data is not uploaded after a specific time period.

36. The method of claim 33 and further comprising:
providing an alternate alert if the data is not uploaded after a specific time period.

37. The method of claim 33 and further comprising:
uploading the data from the memory; and
receiving verification after the data is uploaded from the memory.
